(19) United States
(12) Patent Application Publication
(10) Pub. No.: US 2009/0093686 A1
(43) Pub. Date: Apr. 9, 2009

(54) Multi Automated Severity Scoring

(76) Inventors: Xiao Hu, Los Angeles, CA (US); Neil Martin, Encino, CA (US); Farzad Buxey, Marina Del Rey, CA (US); Vesselin Zlatev, Aliso Viejo, CA (US); Valeriy Nenov, Los Angeles, CA (US)

Correspondence Address: ADELI & TOLLEN, LLP, 11940 San Vicente Blvd, Suite 100, LOS ANGELES, CA, 90049, US

(21) Appl. No.: 12/036,265

(22) Filed: Feb. 24, 2008

Related U.S. Application Data
(60) Provisional application No. 60/978,397, filed on Oct. 8, 2007.

(51) Int. Cl. A61B 5/00

(52) U.S. Cl. 600/300; 600/301

(57) ABSTRACT

Some embodiments of the invention provide an automated method for continuously monitoring at least one patient. The method continuously collects medical data about the patient from several different inputs. The method integrates the collected data, and continuously computes at least one severity score for the patient based on the integrated data. The method uses the severity scores to continuously monitor the patient. In some embodiments, integrating the data includes formatting the data so that that the data can be used to compute the severity score. Formatting the data in some embodiments includes using a set of database tables to convert the data. In some embodiments, the database tables are modifiable by a user. Some embodiments continuously compute multiple severity scores. The severity scores computed in some embodiments include the APACHE II score, the SAPS II score, the MEWS score, or user-defined severity scores.

### CLAIM OF BENEFIT TO PRIOR APPLICATION

This patent application claims the benefit of the U.S. Provisional Patent Application 60/978,397, filed Oct. 8, 2007, entitled “Multi Automated Severity Scoring”.

### FIELD OF THE INVENTION

The invention relates to the field of health care. More specifically, the invention relates to systems and methods for providing multiple objective severity scores and their temporal trends in an automated and configurable fashion, both retrospectively and in real-time.

### BACKGROUND OF THE INVENTION

The quality of health care is constantly evolving and improving as less invasive surgical techniques, more effective medications, and better methods of treatment are constantly being discovered and invented. Improvements in health care have also occurred through better use and management of patient information. One such use has allowed medical personnel to reliably predict future probable conditions of a patient through trend analysis of the patient's information. Trends within various patient vital signs (e.g., blood pressure, heart rate, body temperature, etc.) have been shown to reliably indicate future medical conditions or complications.

Attempts have been made to create a standard or objective way to measure such trends by quantifying the results of such trends into one or more “severity scores”. Severity scores have usually been developed by combined efforts from multiple healthcare organizations. Such efforts have the primary aims of quantifying patient illness such that the mortality rate of an organization can be adjusted by considering the expected survival rate based on these severity scores as well as providing a reliable prognosis of probable changes in the condition of the patient. The severity scores thus assist in providing a quicker response to treat any such changes. To be an objective measure requires that severity scores be defined using patient information that may include laboratory test results, vital signs, etc., as well as clerical information such as age or gender, in come cases.

Many studies have been done on validating existing severity scoring metrics. Severity scores such as Acute Physiology and Chronic Health Examination (APACHE) and Simplified Acute Physiology Score (SAPS) have been well known for purposes including mortality prediction and patient stratification. Other scores, such as the Modified Early Warning Score (MEWS), have been proposed for early detection of patient deterioration and have been validated in several pilot studies. However, the impact of these severity scores into daily clinical practice remains elusive. These severity scores have not been fully accepted and integrated into typical workflows of patient care for possible reasons including lack of an automated scoring system and ambiguities in terms of specification of data collection protocol for scoring. More specifically, barriers to the adoption of such severity scores include insufficient data gathering, time alignment issues resulting from inconsistent data gathering, and improper data processing (e.g., aggregation and unit conversion).

Typically, the data reporting for such severity scoring is conducted on a manual basis by medical personnel assigned the task of gathering and aggregating the required data. As a result, the reporting is at times inconsistent or subjective.

Additional deficiencies in current severity scoring result from the delay associated with the data gathering and analysis. For instance, the existing scoring metrics only take recorded data after it has been manually transcribed from some vital statistic monitor into a database. The time it takes for the data gathering to be completed and further still for the trend analysis to be completed can cause sufficient delay which reduces or defeats the effectiveness and potential early warning provided by the severity score.

The penetration of information technology (IT) into the various aspects of health care has assisted to alleviate some of the data gathering and data management overhead previously associated with providing health care. Establishment and wide adoption of industry-wide standards such as Health Level Seven (HL7) and Digital Imaging and Communications in Medicine (DICOM) together with much improved computational capability, data storage capability, and fast communication platforms, have provided an ideal environment for the further development of more dedicated IT solutions tailored for more specific clinical challenges.

However, there is a need to better leverage information stored and managed by these IT resources to provide improved health care services to patients. Specifically, there is a need for a severity scoring system that: 1) is highly automated and can monitor patients on a continuous basis, 2) supports the computation of multiple scores simultaneously, and 3) supports both retrospective and real-time modes of operation.

The automated system should be flexibly integrated into existing clinical/hospital information systems in order to provide greater access to broader ranges of statistical information. The system either independently or through a separate middleware system should translate patient information from the varying sources into a unified format for use in computing the severity scores. As such, a need exists for the computed results to be consistent irrespective of the means used for data acquisition. Such a system should support the computation of multiple scores simultaneously, therefore providing multiple reference points from which to analyze the condition of a patient or to verify the accuracy of one metric against another. Additionally, there is a need for the data acquisition and trend analysis to occur in near real-time or in real-time so as to provide more effective severity scores allowing for timely responses to any deterioration of a patient's condition.

### SUMMARY OF THE INVENTION

Some embodiments of the invention provide an automated method for continuously monitoring at least one patient. The method continuously collects medical data about the patient from several different inputs. The method integrates the collected data, and continuously computes at least one severity score for the patient based on the integrated data. The method uses the severity scores to continuously monitor the patient. In some embodiments, integrating the data includes formatting the data so that that the data can be used to compute the severity score. Formatting the data in some embodiments includes using a set of database tables to convert the data. In some embodiments, the database tables are modifiable by a user. Some embodiments continuously compute multiple severity scores. The severity scores computed in some embodiments include the APACHE II score, the SAPS II score, the MEWS score, or user-defined severity scores.

### BRIEF DESCRIPTION OF THE FIGURES

FIG. 1 conceptually illustrates a process used by some embodiments to continuously compute and disseminate multiple severity scores for monitoring patients.

FIG. 2 conceptually illustrates a system of some embodiments of the invention for processing patient data.

FIG. 3 conceptually illustrates a process used by some embodiments of the invention to compute at least one severity score for a patient.

FIG. 4 conceptually illustrates a process performed by some embodiments to compute a severity score for a patient at a given time.

FIG. 5 illustrates different elements of the MEWS severity score used by some embodiments.

FIG. 6 conceptually illustrates a process used by some embodiments to select a data point for a particular element of a severity score when computing severity scores in real-time.

FIG. 7 illustrates data points and data selection techniques for real-time scoring.

FIG. 8 conceptually illustrates a process used by some embodiments to select a data point for a particular element of a severity score when computing severity scores retrospectively via batch processing.

FIG. 9 illustrates data points and data selection techniques for retrospective scoring.

FIG. 10 conceptually illustrates a process by which a user alters certain aspects of the system of some embodiments.

FIG. 11 conceptually illustrates a process by which some embodiments of the invention use machine-learning to iteratively optimize a severity score definition.

FIG. 12 conceptually illustrates a computer system of some embodiments.

### DETAILED DESCRIPTION OF THE INVENTION

In the following description, numerous details are set forth for purpose of explanation. However, one of ordinary skill in the art will realize that the invention may be practiced without the use of these specific details. For instance, the techniques described below are described in a specified order, but other embodiments may change the order of the operations while still embodying the current invention.

Some embodiments of the invention provide an automated method for continuously monitoring at least one patient. The method continuously collects medical data about the patient from several different inputs. The method integrates the collected data, and continuously computes at least one severity score for the patient based on the integrated data. The method uses the severity scores to continuously monitor the patient. In some embodiments, integrating the data includes formatting the data so that that the data can be used to compute the severity score. Formatting the data in some embodiments includes using a set of database tables to convert the data. In some embodiments, the database tables are modifiable by a user. Some embodiments continuously compute multiple severity scores. The severity scores computed in some embodiments include the APACHE II score, the SAPS II score, the MEWS score, or user-defined severity scores.

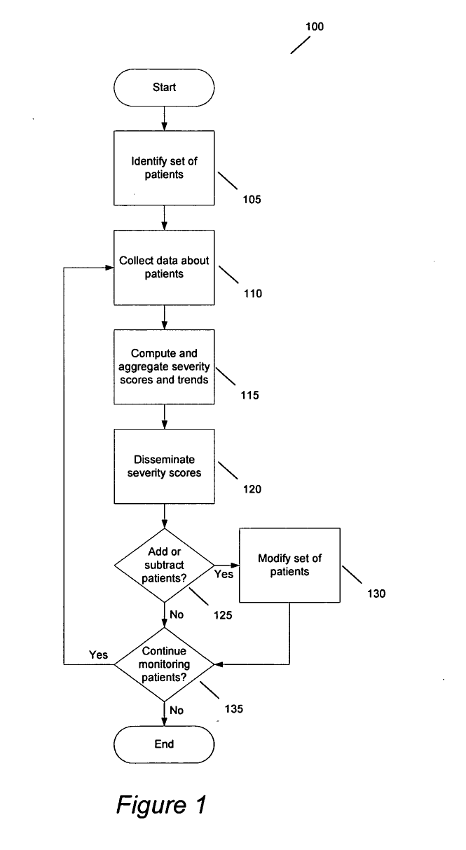

FIG. 1 conceptually illustrates a process 100 by which some embodiments of the invention continuously compute severity scores to monitor patients. The process 100 starts by identifying at 105 a set of patients. The patients might be a set of patients in a hospital or other clinical setting. At 110, the process collects data about the patients. After collecting data, the process computes at 115 severity scores and trends for the patients. Some embodiments that compute multiple severity scores compare the severity scores against each other for validation purposes. Some embodiments aggregate the severity scores to create a composite score (e.g., by assigning different scalar weights to the different severity scores and adding them together). In addition to computing the severity scores, some embodiments compute trends for the severity scores as a change in the severity score over time.

After computing and aggregating severity scores, the process 100 disseminates at 120 the computed severity scores for the patients. In some embodiments, the severity scores may be well-known scores or user-defined scores. The trends in computed scores are displayed in some embodiments to health care professionals in charge of monitoring the patients. In some embodiments, the health care professionals may use the computed severity scores to predict mortality, prioritize patient care, or issue alerts to provide rapid care for specific patients. The process then determines at 125 whether to add or subtract any patients from the identified set of patients. If the process is being used to continuously monitor patients in an ICU, then when patients are brought into or discharged from the ICU, they need to be added to or subtracted from the set of patients being continuously monitored. If the process determines that patients need to be added or subtracted, the process modifies at 130 the set of patients being monitored. The process then determines at 135 whether to continue monitoring the set of patients. If the process determines not to continue monitoring the set of patients, the process ends. If the process determines to continue monitoring the set of patients, the process returns to 110 and performs operations 110-135 again. The process will compute and disseminate severity scores until it determines to no longer continue monitoring patients.

### I. System for Computing Severity Scores

B. Overall System of Some Embodiments

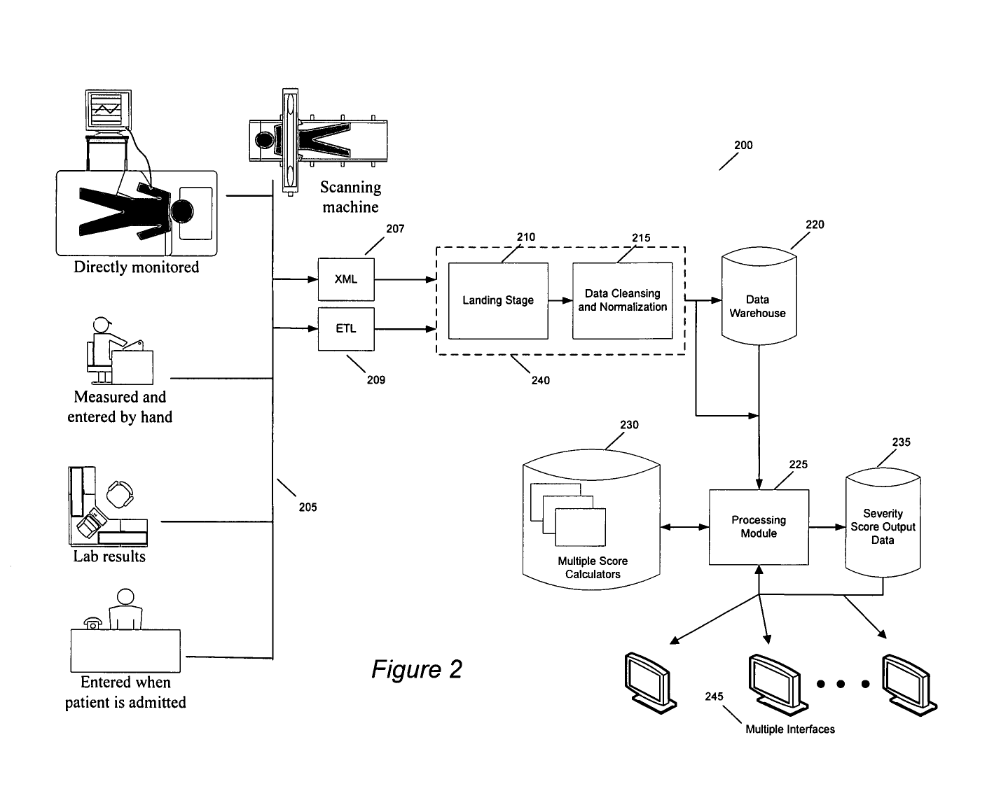

FIG. 2 conceptually illustrates a clinical information system 200 of some embodiments of the invention that can perform process 100. Patient data 205 is received from several patient data sources at landing stage 210. Patient data 205 can gathered from a variety of sources such as (1) real-time monitors at the patients (2) scans such as MRIs, (3) lab results, (4) clerical data (e.g. patient age and gender) entered upon admission to the hospital, or (5) other patient information. In some embodiments, the patient data is HL7, DICOM, or Nursing data. Patient data 205 may arrive at landing stage 210 via a variety of processes, including XML processes 207 (such as the Simple Object Access Protocol) and extract-transform-load (ETL) processes 209. From landing stage 210, the patient data in some embodiments is cleansed and normalized at data cleansing module 215. Once the data is cleansed and normalized, it is stored in a data warehouse 220 (for example, an SQL server data warehouse). In some embodiments, the data warehouse includes multiple data storages.

In some embodiments, the landing stage 210 and data cleansing module 215 are modules of middleware system 240. Some embodiments integrate with multiple middleware systems 240 that each have a landing stage 210 and data cleansing module 215.

A processing module 225 can pull data from the data warehouse for processing. In some embodiments, the processing module 225 receives data in real time directly from the middleware system 240. In some embodiments, the processing module 225 performs a normalization process on the received data to prepare the data for input into severity score calculators 230. Severity score calculators 230 are sets of rules the processing module 225 uses to compute severity scores for patients from the patient data received from the data warehouse 220. In some embodiments, the severity score calculators 230 are stored in the same data warehouse 220 as the patient data. The severity score calculators 230 of some embodiments provide rules for calculating known severity scores such as APACHE II, SAPS II, and MEWS. In some embodiments, some of the severity score calculators 230 might be user-defined to provide rules for calculating a severity score defined by the user. Further, each severity score calculator 230 provides rules for data selection to determine which data to use in calculating the severity scores. The severity score calculator rules in some embodiments are a set of “if-then” statements. The processing module 225 uses the calculators 230 to compute at least one severity score from the patient data, and sends the severity score data to a storage 235. From the storage 235, the severity score output data can be used for display, analysis, or other uses. In some embodiments, the storage 235 is the same data warehouse that stores the patient data, the severity score calculators 230, or both. In some embodiments, the severity score output data is fed back into the processing module for trend computation, machine-learning, or other purposes.

In some embodiments, the system also includes multiple interfaces 245. Interfaces 245 can be computer monitors and input terminals as well as thin client devices such as PDAs or cell phones. Interfaces 245 can receive severity score data either directly from the processing module 225 or from the storage 235 that stores the severity score output data. The interfaces 245 can display severity score information about patients, including both absolute severity scores and changes to a patient's severity score. The interfaces 245 can also display the patient data 105. Interfaces 245 can also be used by some users of the system to alter characteristics of the severity score calculators or of the data integration process.

In some embodiments of the invention one or more of the data collection, data integration, and score computation processes are automated. In some embodiments, all of these processes are automated. The processes are automated in that the patient data 205 is continuously arriving at the data warehouse 220, and at regularly scheduled intervals, with no user intervention required, the processing module 225 retrieves the patient data from the data warehouse 220, integrates the data for computation, and uses the multiple severity score calculators 230 to compute severity scores. Some severity scores might be computed at different time intervals than other severity scores. For example, some embodiments might compute a first severity score every 15 minutes, while a second severity score would be computed every hour.

B. Integration with Middleware Systems

As mentioned above, the processing module 225 must integrate the received data so that it can be understood by the severity score calculators, whether from the data warehouse 220 or directly from the middleware system 240. The integration in some embodiments is done with the use of database tables that allow for easy integration with multiple middleware systems. In some embodiments, database tables are used to specify how a specific input, such as the heart rate from a specific vendor's heart rate monitor, maps to a severity score element that assigns an element score for heart rate (see Section II for discussion on how such element scores are calculated). In some embodiments, these database tables might specify where in the received data the processing module 225 can find the patient identification for the piece of data, the value of the piece of data (e.g., the measured heart rate), etc. In some embodiments, the database tables also specify how to map this received data into data that can be used by a severity score calculator.

### II. Methods for Computing Multiple Severity Scores

A. Process for Computing Multiple Severity Scores

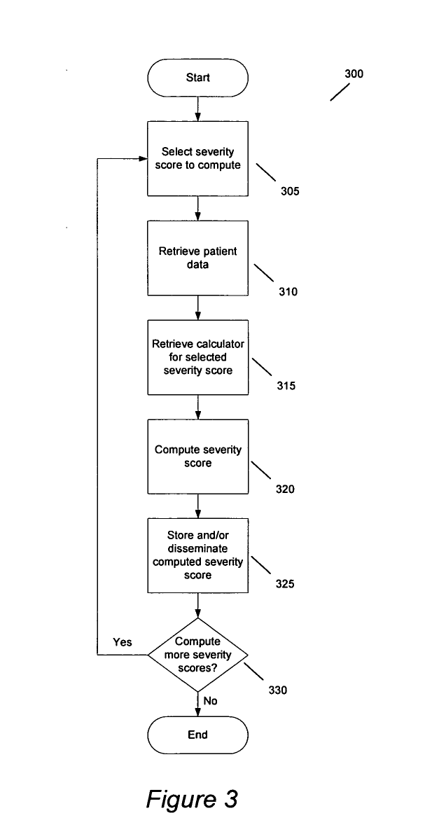

FIG. 3 conceptually illustrates the process 300 used by some embodiments of the invention to compute at least one severity score for at least one patient. Some embodiments use the process 300 to compute multiple severity scores for multiple patients, a single severity score for multiple patients, or multiple severity scores for a single patient. Some embodiments compute severity scores continuously. In some embodiments, continuous computation means the severity scores are computed at regular intervals with no human intervention. The processing module 225 of system 200 computes the at least one severity score in some embodiments.

At 305, the process 300 selects a severity score to compute for a patient. In some embodiments, the process 300 selects a severity score that needs to be computed at regular time intervals. For example, if a severity score is being computed every fifteen minutes, and the last time the severity score was computed was fifteen minutes prior, the process 300 might select at 305 that severity score to compute. Some embodiments compute severity scores retrospectively in a batch processing mode. In such embodiments, a severity score might be computed for several different times all at once. Some embodiments calculate scores both retrospectively and in real-time.

After selecting a severity score at 305, the process 300 retrieves (at 310) patient data. In some embodiments, this patient data is retrieved from the data warehouse 220. In some embodiments, the process 300 only retrieves the patient data relevant to the selected severity score. For example, the MEWS severity score only has five components (heart rate, respiratory rate, blood pressure, temperature, and AVPU score), so some embodiments retrieve data at 310 for only these five components when the MEWS score is the severity score selected at 305. In some embodiments, retrieving patient data includes integrating that data for use by a severity score calculator.

At 315, the process 300 retrieves the calculator for the selected severity score. Each calculator includes a set of rules that defines how to compute the selected severity score. In some embodiments, each calculator also includes rules for data selection. After retrieving patient data at 310 and the selected severity score calculator at 315, the process 300 can compute the selected severity score at 320. The process 300 computes the selected severity score by applying the rules defined in the severity score calculator to the patient data.

At 325, the process stores and/or disseminates the computed severity score. In some embodiments, the computed severity score is stored in storage 235. In some embodiments, the computed severity score is output to one or more interfaces 245. After storing and/or disseminating the computed severity score at 325, the process 300 determines at 330 whether to compute more severity scores. In some embodiments of the invention that continuously generate multiple severity scores in real-time, the process determines at 330 whether more scores need to be calculated for the present time. The process might need to compute the same severity score for another patient or a second severity score for the same patient. In some embodiments that generate at least one severity score retrospectively with batch processing, the process determines at 330 whether the same severity score needs to be calculated for a different time, or whether different severity scores need to be calculated. If more severity scores need to be computed, the process returns to 305 and selects another severity score to compute. If no more severity scores need to be computed, the process ends.

B. Process for Computing a Severity Score

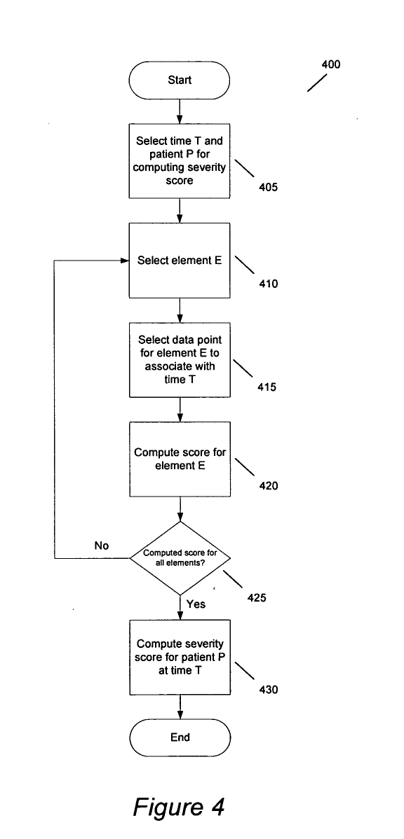

FIG. 4 conceptually illustrates a process 400 performed by some embodiments of the invention to compute a severity score for a patient at a given time. In some embodiments, process 400 corresponds to operation 320 of process 300 and is performed by processing module 225 of system 200. Process 400 starts by selecting at 405 a time T and a patient P for which the severity score will be computed. In some embodiments, the time T and patient P are inputs to the process, and the processing module 225 has already retrieved data about patient P. At 410, the process 400 selects a first element E. Elements are the individual components of a severity score.

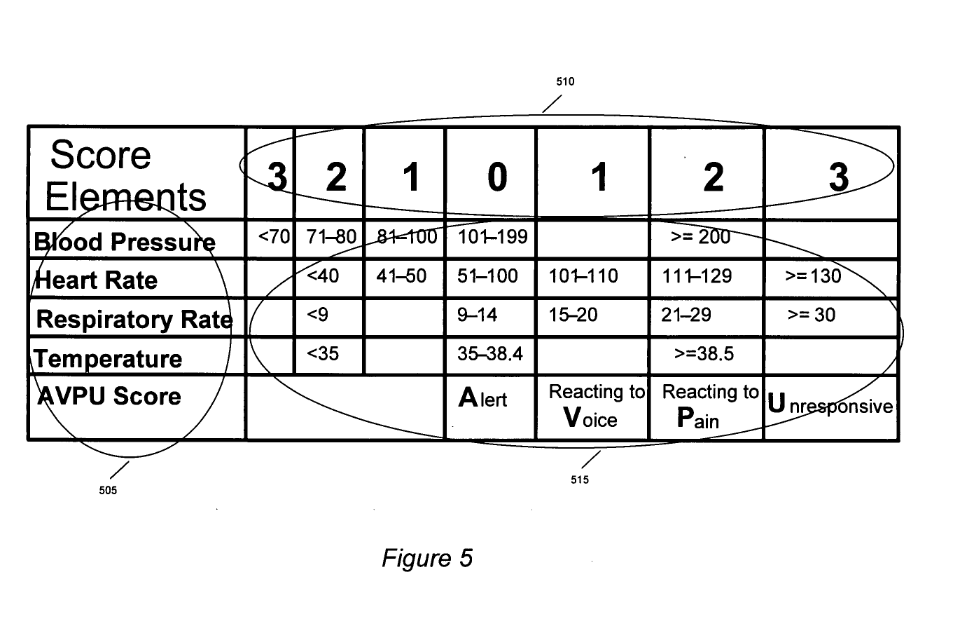

FIG. 5 illustrates different elements 505 of the MEWS severity score used by some embodiments. FIG. 5 illustrates elements 505, element scores 510, and ranges 515. The elements 505 for the MEWS score are blood pressure, heart rate, respiratory rate, temperature, and AVPU score. Thus, if computing a MEWS score for patient P, the process 400 selects at 410 one of the five elements 505, e.g. heart rate. Other scores might have more or fewer elements than the MEWS score. For example, the APACHE II score has 12 primary elements.

After selecting an element E, the process 400 selects at 415 a data point for element E to associate with the time T for which the severity score is being computed. The process 400 might use one of a number of techniques such as selecting the most recent data point, selecting the most severe data point in a time window around time T, or other techniques. Data selection techniques are discussed in greater detail in subsections II.C and II.D below.

Once a data point is selected for element E, the process 400 computes at 420 a score for element E. In the severity scoring definitions of some embodiments, a score for an individual element is an integer. In the case of MEWS, each element 505 is assigned a score from 0 to 3. The scores are shown as 510 in FIG. 5. The process 400 computes a score for element E by determining the range into which the data point selected at 415 falls. The ranges for the different elements of the MEWS score are shown as 515 in FIG. 5. In the case of the heart rate element, a score of zero is computed if the heart rate data point selected is from 51-100 beats per minute, as this is indicative of decent health. A heart rate of 41-50 or 101-110 beats per minute results in an element score of one, a heart rate of 111-129 or less than 40 beats per minute results in an element score of two, and a heart rate of greater than 130 beats per minute results in an element score of three. In the case of MEWS, and most severity scores, higher scores are indicative of more serious patient condition. However, some severity scores might use lower scores or greater distance from a baseline value to indicate more serious patient condition.

After computing the score for element E, the process 400 determines at 425 whether the element score for all elements of the severity score have been computed. If more elements remain, the process 400 returns to 410, selects another element E, and repeats operations 415 and 420 for the newly selected element E. If element scores have been computed for all elements of the severity score, then the process computes the severity score at 430. In some embodiments, the severity score is computed as the sum of all the individual scores. In the case of the MEWS score, the severity score can be from zero to fourteen (the temperature element can not have a value of three, as shown in FIG. 5). In some embodiments, the severity score is calculated differently, for example by weighting the various element scores.

C. Computing Severity Scores in Real-Time

Some embodiments of the invention compute at least one severity score in real-time. Some such embodiments continuously receive patient data from multiple sources. Some embodiments compute multiple severity scores in real-time. Some embodiments compute severity scores at specified time intervals. Some embodiments that compute multiple severity scores compute a first score at a first time interval and a second score at a second time interval. For example, some embodiments compute a first score every fifteen minutes and a second score every hour.

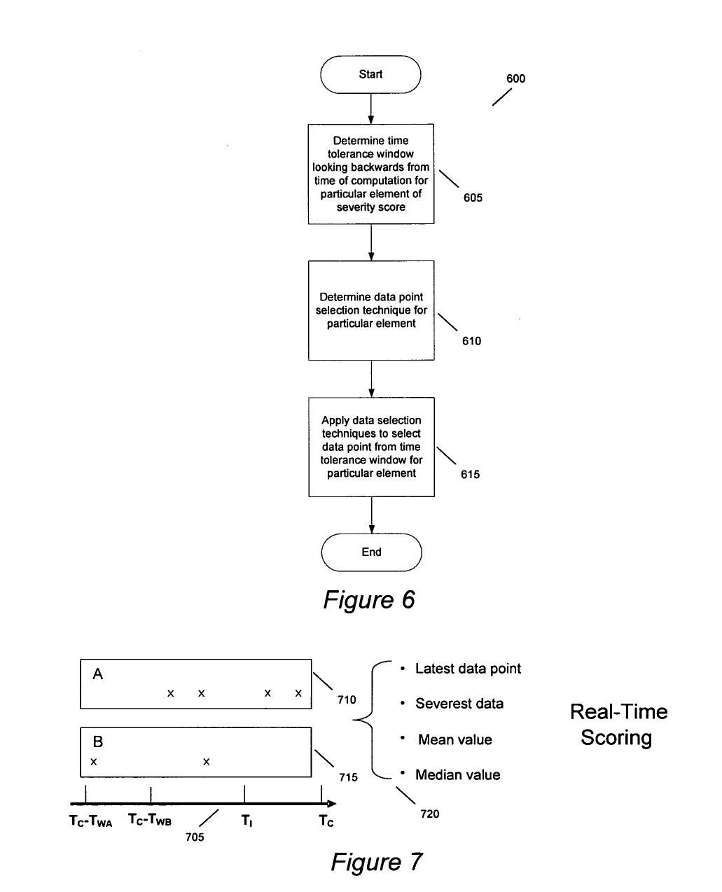

Referring to process 400 of FIG. 4, the time T for which the severity score is computed is always the time of computation when computing scores in real-time. FIG. 6 conceptually illustrates process 600 that some embodiments of the system use to select a data point for a particular element of a severity score when computing severity scores in real-time. In some embodiments, the process 600 corresponds to the data selection operation that process 400 performs at 415. FIG. 7 provides an illustration of data points and data selection techniques used by some embodiments for real-time scoring. FIG. 7 illustrates time axis 705, data points 710 for element A, data points 715 for element B, and data selection techniques 720.

Time axis 705 shows four time points: T_{C}, T_{I}, T_{C}-T_{WA}, and T_{C}-T_{WB}. T_{C} represents the present time for which the severity score is being calculated. T_{I} is the time at which the severity score was previously calculated. Thus, if the severity score is calculated every hour, then T_{I} is one hour prior to T_{C}. Some embodiments check to see if there are any new data points since T_{I}, and if there are no new points for any of the elements, output the score for T_{C} as the score computed at T_{I}. However, because the range of data points may have changed due to the use of tolerance windows, some embodiments recompute the severity score even if there are no new data points for any elements. T_{WA} specifies the tolerance window for element A, and T_{WB} specifies the tolerance window for element B. The tolerance window T_{WA} for element A is the amount of time around T_{C} from which data points can be selected when computing an element score for element A. The tolerance window for element B is defined similarly. In some embodiments, the tolerance window for all elements is the same. However, in other embodiments, different elements of a severity score will have different tolerance windows.

At 605, the process 600 determines a time tolerance window for a particular element of the severity score being computed. In some embodiments, the tolerance window for each element is part of the severity score definition provided in the severity score calculators 230. In real-time scoring, since T_{C} is the time of computation, only the tolerance windows looking backwards in time are relevant. Thus, for element A, data can be selected from any point in the time window from T_{C}-T_{WA} to T_{C}. In the example shown in FIG. 7, there are four data points 710 to choose from for element A. There is only one data point 715 to choose from for element B, because the other data point falls outside the time tolerance window T_{WB} for element B. In some embodiments, however, the number of data points in the time tolerance window will be very large.

At 610, the process 600 determines a data point selection technique for the particular element. In some embodiments, the data point selection technique for each element is also part of the severity score definition provided in the severity score calculators 230. Some embodiments use a variety of techniques 720 to select a data point for an element. Some embodiments use the same technique for each element of a severity score, while other embodiments use different techniques for some or all elements. Some embodiments simply use the most recent data point, while some use a mean value or median data point. Some embodiments select the most severe data point from the time tolerance window. For some elements, the most severe data point might be the highest data point, for some elements the most severe data point might be the lowest data point, and for some elements the most severe data point might be the furthest from a baseline normal value, whether high or low. Some embodiments perform standard digital signal processing techniques such as a Fast Fourier Transform (FFT) or other techniques on the data before selecting a data point so as to eliminate noise. In the case of heart rate, for example, noise might come from an ECG lead falling off of a patient.

At 615, the data point selection process 600 applies the chosen data selection technique to the data from the time tolerance window for the particular element in order to select a data point for the particular element. Once a data point is selected for an element, the score for the element can be computed as described above at operation 420 of process 400. Real-time scoring can be used by health care professionals for a variety of purposes, such as prioritization of rounding lists and alarm generation for rapid response teams. These uses will be discussed further in Section III below.

D. Computing Scores Retrospectively via Batch Processing

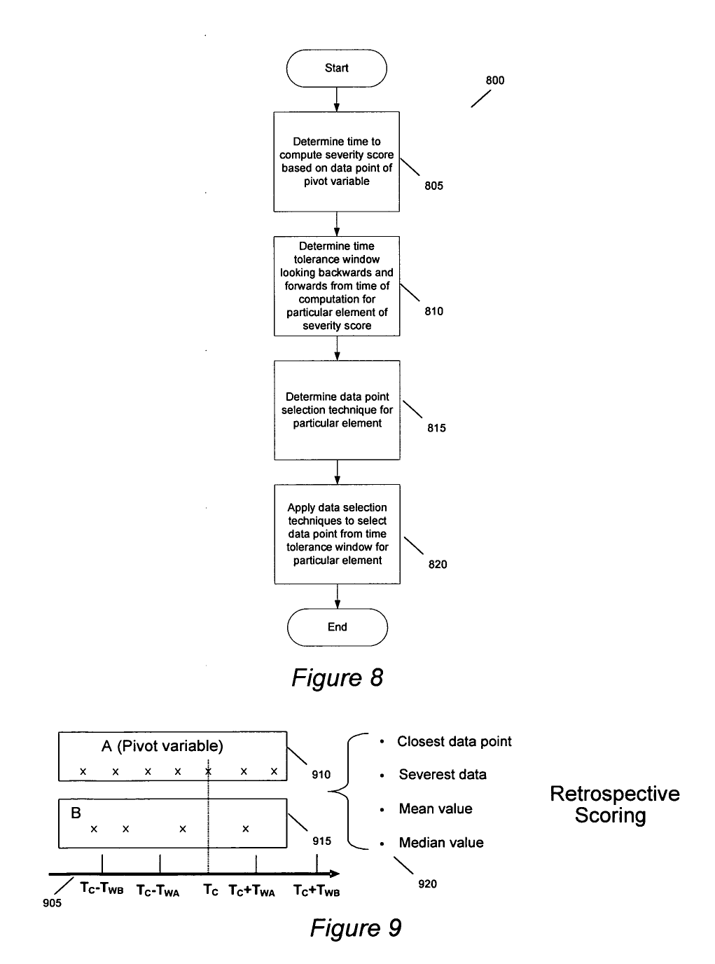

Some embodiments of the invention compute scores retrospectively via batch processing. FIG. 8 conceptually illustrates process 800 that some embodiments use to select a data point for a particular element of a severity score when computing severity scores retrospectively via batch processing. In some embodiments, the process 800 corresponds to the data selection operation that process 400 performs at 415. FIG. 9 provides an illustration of data points and data selection techniques used by some embodiments for retrospective scoring. FIG. 9 illustrates time axis 905, data points 910 for element A, data points 915 for element B, and data selection techniques 920. When computing severity scores retrospectively, some embodiments select one element as a pivot variable. The retrospective scoring process of some embodiments computes a severity score for each time for which there is a data point for the pivot variable In some embodiments, the pivot variable is the most frequently measured element of the severity score being computed. In FIG. 9, A is the pivot variable because it is more frequently measured than element B. Generally, the pivot variable will be an element like heart rate that is measured continuously through a real-time monitor. In some embodiments, the pivot variable might be a variable that is less frequently measured but is more important to severity score calculation, although choosing a less frequently measured pivot variable may result in loss of information.

At 805, process 800 determines a time to compute a severity score based on a data point for the pivot variable. In FIG. 9, time axis 905 shows five time points. T_{C} represents the present time for which the severity score is being calculated, which is set to a time point corresponding to a measurement of the pivot variable. T_{WA} specifies the tolerance window for element A, and T_{WB} specifies the tolerance window for element B. The tolerance window T_{WA} for element A is the amount of time around T_{C} from which data points can be selected when computing an element score for element A. The tolerance window for element B is defined similarly. In some embodiments, the tolerance window for all elements is the same. However, in other embodiments, all different elements of a severity score will have different tolerance windows. In some embodiments, the tolerance window for the pivot variable will be zero.

At 810, the process 800 determines a time tolerance window for a particular element of the severity score being computed. In some embodiments, the tolerance window for each element is part of the severity score definition provided in the severity score calculators 230. In retrospective scoring, since T_{C} can be in the middle of the time for which data points are available, tolerance windows looking both forwards and backwards in time are relevant. Thus, for element A, data can be selected from any point in the time window from T_{C}-T_{WA} to T_{C}+T_{WA}. In the example shown in FIG. 9, there are three data points 910 to choose from for the pivot variable, element A. There are also three data points 915 to choose from for element B. In some embodiments, however, the number of data points in the time tolerance window will be very large.

At 815, the process 800 determines a data point selection technique for the particular element. In some embodiments, the data point selection technique for each element is also part of the severity score definition provided in the severity score calculators 230. Some embodiments use a variety of techniques 920 to select data points for elements. Some embodiments use the same technique for each element of a severity score, while other embodiments use different techniques for some or all elements. Some embodiments use the closest data point (always the data point at time T_{C} for the pivot variable), while some use a mean value or median data point. Some embodiments select the most severe data point from the time tolerance window. For some elements, the most severe data point might be the highest data point, for other elements the most severe data point might be the lowest data point, and for some elements the most severe data point might be the furthest from a baseline normal value, regardless of whether high or low. Some embodiments perform standard digital signal processing techniques such as a Fast Fourier Transform (FFT) or other techniques on the data before selecting a data point so as to eliminate noise.

At 820, the data point selection process 800 applies the data selection technique to the data from the time tolerance window for the particular element in order to select a data point for the particular element. Once a data point is selected for an element, the score for the element can be computed as described above at operation 420 of process 400. Retrospective scoring can be used to examine the usefulness of different severity scores as predictors of patient outcomes. The ability to compute multiple severity scores simultaneously allows users to examine which severity scores do a better job of predicting outcomes such as mortality, cardiac arrest, or ICU discharge. Examining the successfulness of a severity score can allow users to alter the severity score by changing the elements, the ranges, or the data selection processes for the severity score.

III. Monitoring Patients with Severity Scoring

Some embodiments of the invention use the continuous real-time computation of one or more severity scores for real-time monitoring of patient health in a hospital or other clinical setting. Some embodiments provide, at an interface 245, a list of patients along with computed severity scores and trends in the computed severity scores. A trend is a change in the severity score over time. Users, such as physicians making rounds in a hospital, can use the computed severity scores or trends to prioritize patient rounding lists. For example, a user can sort by the trend of a specific severity score such that the patient list would start with the patients whose severity score has increased the most over the last time interval. Some embodiments of the severity scoring system alert medical care providers, such as rapid response teams, in an automated fashion based on the values or trends in one or more computed severity scores. Some embodiments are used for hospital management purposes such as bed control, discharge planning, or nurse allocation. These real-time monitoring processes are described in detail in the concurrently filed application with the title “Patient Monitoring”, having attorney docket no. GCQI.P0007, which is incorporated herein by reference.

In some embodiments, the severity scores are used to intelligently recommend a display (or “dashboard”, where a dashboard is a specific collection of window panes with patient information) to a user for monitoring a specific patient. In these embodiments, a user examining a patient list at an interface 245 selects a patient. The selection of a patient triggers the system to determine, based on a computed severity score for the patient or at least one element of a severity score, what window panes should be shown to the user in a dashboard. This process is described in detail in the concurrently filed application with the title “Intelligent Dashboard”, having attorney docket no. GCQI.P0008, which is incorporated herein by reference.

### IV. User Feedback and Input

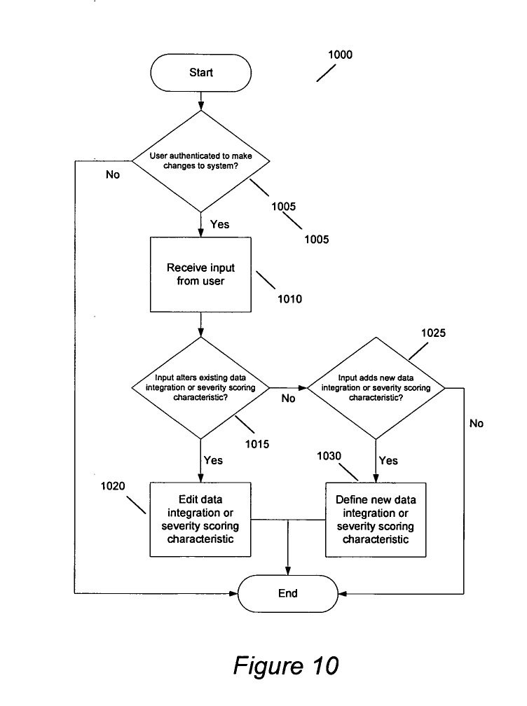

In some embodiments, a user can alter aspects of the severity scoring system. Specifically, in some embodiments, users can provide input that either alters existing data integration or severity scoring characteristics or adds a new data integration or severity scoring characteristic. FIG. 10 conceptually illustrates a process 1000 by which a user alters these aspects of the clinical information system. First, the process determines at 1005 whether a user is authenticated to alter aspects of the severity scoring system. Authentication can be done by any standard process, such as mandating that users login to the system and assigning different administrative capabilities to different users. If the user is not authenticated to alter aspects of the severity scoring system, the process 1000 ends. If the user is authenticated, the process 1000 proceeds to 1010, where the process receives input from the user. In some embodiments, the input can be a new severity score definition, an alteration to an existing severity score definition, a new database table definition for integration with middleware, feedback regarding severity score output, or other input.

After receiving input at 1010, the process 1000 determines (at 1015) whether the input alters an existing data integration or severity scoring characteristic. In some embodiments, a data integration or severity scoring characteristic can be directly altered by a user editing database tables. In some embodiments, a user provides feedback regarding severity score output, and the severity scoring system processes this feedback to determine whether an existing severity scoring characteristic should be altered. If the process determines that the input alters an existing data integration or severity scoring characteristic, the process 1000 proceeds to 1020. At 1020, the process edits the data integration or severity scoring characteristic as required by the input, then ends. If the process determines at 1015 that the input does not alter an existing data integration or severity scoring characteristic, the process moves to 1025.

At 1025, the process 1000 determines whether the input adds a new data integration or severity scoring characteristic. In some embodiments, a data integration or severity scoring characteristic can be directly added by a user adding new database tables. If the input does not add a new data integration or severity scoring characteristic, the process ends. If the input adds a new data integration or severity scoring characteristic, the process proceeds to 1030 and defines the new data integration or severity scoring characteristic as required by the input, then ends.

A. Adding or Altering Data Integration Characteristics

As discussed in Section I.B, the system of some embodiments uses database tables to integrate data from middleware systems. If the system is going to receive data from a new type of data input, such as a previously unused type of heart rate monitor, then a user would need to define the data integration characteristics for data from the new heart rate monitor. To do so, a user can input (at 1010 of the process 1000) new definitions in some embodiments for the required data integration characteristics. In some embodiments, these new definitions are in the form of database tables that specify how to map the received data to an element of a severity score. For example, a user might need to specify where the relevant pieces of information (patient identifier, data value, etc.) can be found in the received data, and how to convert the data for use by a severity score calculator (e.g., any unit conversion that is needed).

B. Defining or Directly Editing Severity Scoring Characteristics

In some embodiments, a user can directly edit existing severity scoring characteristics or define a new severity score. Similar to adding or altering data integration characteristics, adding or altering severity scoring characteristics is performed in some embodiments when input is received adding or altering database tables. Database tables are used by some embodiments to define a severity scoring metric. Database tables provide the elements of a severity score, define how each element of a score is calculated, and define how the elements are combined to compute a severity score. In some embodiments, the database tables provide, for each element, a data selection procedure (time tolerance window and data point selection technique) and the different data value ranges and corresponding element scores. By editing these database tables, a user can alter an existing severity scoring technique. For example, a user might decide to change a time tolerance window for a particular element of a severity score, and could directly edit the database table to do so. Some embodiments of the invention present a user interface which allows an authenticated user to select an element to edit, and provides the ability to edit that element without the user being required to directly edit the database table.

In some embodiments, a user bases the decision to edit a severity score on previous severity scoring results. For example, a user can look at the outcomes for a set of patients (e.g., whether the patient was discharged or transferred from ICU, whether the patient suffered a catastrophic event such as death or cardiac arrest, etc.) along with severity scores for the set of patients for a time period leading up to the patient outcomes. A user can produce this data by using some embodiments of the invention to perform retrospective scoring for the set of patients in question. The user can analyze the severity score data and the patient outcomes to determine how to optimize the severity score definitions or the data selection techniques for better predictions when using the system for real-time monitoring as described in Section III.

In some embodiments, a user can also add new severity score definitions. A user might have studied data and determined that a different set of elements provides a severity score that best predicts the likelihood of patient mortality, cardiac arrest, or other such catastrophic event. In embodiments that store score definitions in database tables, a user can input a new set of database tables to define the elements of a new severity score, how each element score is calculated, and how the elements are combined to calculate the severity score.

C. Machine Learning

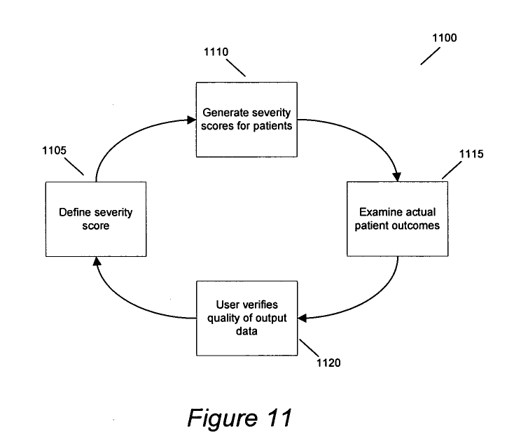

In some embodiments, a user verifies severity score output data against actual patient outcomes, and the severity scoring system uses machine-learning to optimize at least one severity score definition. FIG. 11 conceptually illustrates a process 1100 by which some embodiments of the invention use machine-learning to iteratively optimize a severity score definition. At 1105, a severity score is defined. In the first iteration, the severity score is defined by a user, or is a commonly-used severity score definition such as MEWS, APACHE II, or SAPS II. At 1110, severity scores are generated, either in real-time or retrospectively, for a set of patients. Ideally, the severity scores should be predictive of patient outcomes. The patients will have actual outcomes, and at 1115 a user, such as a physician, examines the actual patient outcomes.

At 1120, the user provides feedback to the severity scoring system as to whether the severity scores were accurately predictive of the eventual patient outcomes. For example, if a patient's severity score started trending upwards, indicating a high likelihood of a catastrophic event, and the patient instead was discharged from the ICU that day, the user would indicate that the severity score failed to accurately predict the patient outcome. The process then returns to 1105, and the severity scoring system can use the feedback to determine how the severity score definition can be changed to better predict patient outcomes. For example, the system might alter the time tolerance window for one or more elements of the severity score, edit the element score ranges for one or more elements of the severity score, or add or eliminate an element from the severity score definition. The more feedback the system receives, the more data it has to work with, and the better the severity score definitions will become.

### V. Computer System

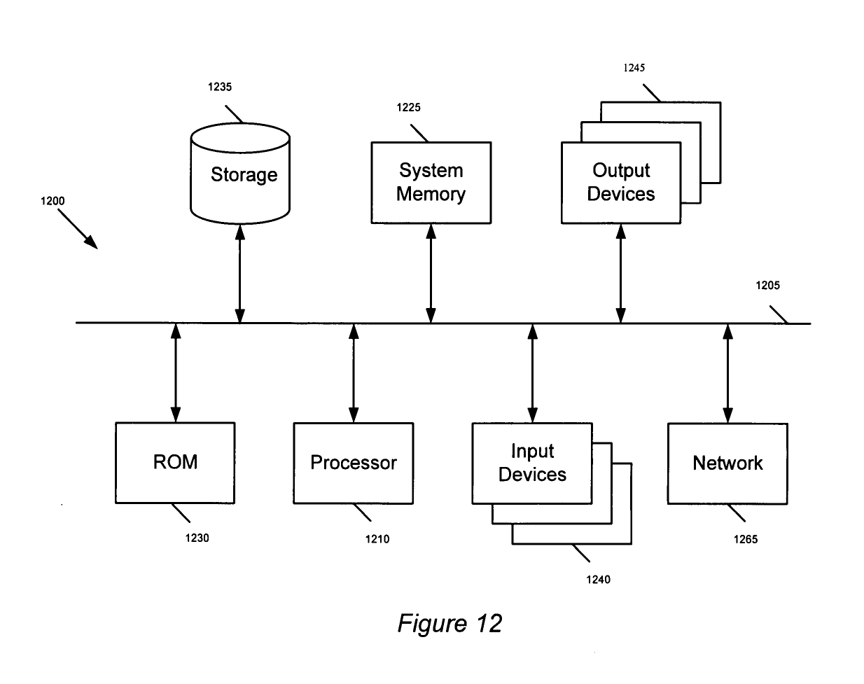

FIG. 12 illustrates a computer system with which some embodiments of the invention are implemented. Computer system 1200 includes a bus 1205, a processor 1210, a system memory 1225, a read-only memory 1230, a permanent storage device 1235, input devices 1240, and output devices 1245.

The bus 1205 collectively represents all system, peripheral, and chipset buses that communicatively connect the numerous internal devices of the computer system 1200. For instance, the bus 1205 communicatively connects the processor 1210 with the read-only memory 1230, the system memory 1225, and the permanent storage device 1235.

The various memory units 1225, 1230, and 1235 are parts of the computer system's 1200 computer readable medium from which the processor 1210 retrieves instructions to execute and data to process in order to execute the processes of the invention. The read-only-memory (ROM) 1230 stores static data and instructions that are needed by the processor 1210 and other modules of the computer system. The permanent storage device 1235, on the other hand, is a read-and-write memory device. This device is a non-volatile memory unit that stores instructions and data even when the computer system 1200 is off. Some embodiments of the invention use a mass-storage device (such as a magnetic or optical disk and its corresponding disk drive) as the permanent storage device 1235.

Other embodiments use a removable storage device (such as a floppy disk or USB flash disk) as the permanent storage device. Like the permanent storage device 1235, the system memory 1225 is a read-and-write memory device. However, unlike storage device 1235, the system memory is a volatile read-and-write memory, such a random access memory. The system memory stores some of the instructions and data that the processor needs at runtime. In some embodiments, the invention's processes are stored in the system memory 1225, the permanent storage device 1235, and/or the read-only memory 1230.

The bus 1205 also connects to the input and output devices 1240 and 1245. The input devices enable the user to communicate information and select commands to the computer system. The input devices 1240 include alphanumeric keyboards and pointing devices. The output devices 1245 display images generated by the computer system. For instance, these devices display a graphical user interface. The output devices include printers and display devices, such as cathode ray tubes (CRT) or liquid crystal displays (LCD).

Finally, as shown in FIG. 12, bus 1205 also couples computer 1200 to a network 1265 through a network adapter (not shown). In this manner, the computer can be a part of a network of computers (such as a local area network (“LAN”), a wide area network (“WAN”), or an Intranet, or a network of networks, such as the internet For example, the computer 1200 may be coupled to a web server (network 1265) so that a web browser executing on the computer 1200 can interact with the web server as a user interacts with a graphical user interface that operates in the web browser.

Any or all components of computer system 1200 may be used in conjunction with the invention. For instance, each of the computer readable memories of the computer system 1200 may function as one or more of the storages for some embodiments of the invention. One of ordinary skill in the art would appreciate that any other system configuration may also be used in conjunction with the present invention.

While the invention has been described with reference to numerous specific details, one of ordinary skill in the art will recognize that the invention can be embodied in other specific forms without departing from the spirit of the invention. Thus, one of ordinary skill in the art would understand that the invention is not to be limited by the foregoing illustrative details, but rather is to be defined by the appended claims.

We claim:

1. An automated method of continuously monitoring at least one patient, the method comprising:
a) continuously collecting medical data about the patient from a plurality of different inputs;
b) continuously computing at least one severity score for the patient based on the integrated data;
c) using the computed severity scores to continuously monitor the patient.

2. The method of claim 1, wherein computing the at least one severity score comprises formatting the data from the plurality of different inputs so that the data can be used to compute the at least one severity score.

3. The method of claim 2, wherein formatting the data comprises using a set of database tables to convert the data.

4. The method of claim 1, wherein one of the inputs is a real-time heart rate monitor.

5. The method of claim 1, wherein one of the inputs is a lab report stored on a hospital information system.

6. The method of claim 1, wherein multiple severity scores are continuously computed.

7. The method of claim 1, wherein one of the severity scores is the APACHE II score.

8. The method of claim 1, wherein one of the severity scores is the SAPS II score.

9. The method of claim 1, wherein one of the severity scores is the MEWS score.

10. The method of claim 1, wherein one of the severity scores is user-defined.

11. The method of claim 10, wherein a user defines the user-defined severity score by defining a set of database tables.

12. The method of claim 1 further comprising receiving user feedback about the at least one severity score.

13. The method of claim 12 further comprising modifying the at least one severity score based on the user feedback.

14. The method of claim 13, wherein the severity score is modified by machine-learning.

15. The method of claim 13, wherein the severity score is modified by a user.

16. A method for assessing a patient, the method comprising:
a) receiving medical data about the patient from a set of data sources, wherein the medical data comprises data points from a plurality of times;
b) for each data source, selecting a data point to associate with a particular time; and
c) computing at least one severity score for the particular time based on the selected data points.

17. The method of claim 16, wherein computing a severity score comprises:
a) mapping each data point to an integer score; and
b) summing the integer scores to compute the severity score.

18. The method of claim 16, wherein computing at least one severity score comprises computing multiple severity scores.

19. The method of claim 16, wherein for a particular data source, the data point selected is the lowest data point for a specified amount of time prior to the particular time.

20. The method of claim 19, wherein data points resulting from noise are not selected.

21. The method of claim 16, wherein for a particular data source, the data point selected is the highest data point for a specified amount of time prior to the particular time.

22. The method of claim 16, wherein the medical data is received and the at least one severity score is computed in real-time.

23. The method of claim 16, wherein selecting a data point for a particular data source comprises utilizing user feedback to determine the optimal data point to select.

24. The method of claim 16, wherein the medical data comprises non-real-time data, and the at least one severity score is computed retrospectively.
